(19) United States
(12) Patent Application Publication
(10) Pub. No.: US 2002/0182151 A1
(43) Pub. Date: Dec. 5, 2002

(54) Benzoylalkylindolepyridinium componds and pharmaceutical compositions comprising such compounds

(75) Inventors: William G. Rice, Madison, CT (US); Mingjun Huang, Rockville, MD (US); Robert W. Buckheit JR., Myersville, MD (US); David G. Covell, Chevy Chase, MD (US); Grzegorz Czerwinski, Middletown, DE (US); Christopher J. Michejda, North Potomac, MD (US); Vadim Makarov, Moscow (RU)

Correspondence Address: KLARQUIST SPARKMAN, LLP, One World Trade Center, Suite 1600, 121 S.W. Salmon Street, Portland, OR, 97204, US

(73) Assignee: The Government of the U.S.A. as represented by the Secretary of the Dept. of Health & Human Services

(21) Appl. No.: 10/017,323

(22) Filed: Dec. 13, 2001

Related U.S. Application Data
(60) Provisional application No. 60/256,556, filed on Dec. 18, 2000.

(51) Int. Cl. A61K 49/00; C07D 471/02; A61K 31/4745

(52) U.S. Cl. 424/9.6; 514/291; 546/87

(57) ABSTRACT

The design, synthesis and antiviral activity of certain antiviral compounds are disclosed examples of which are shown below. These compounds inhibit the reverse transcriptase enzymes of several retroviruses, including human immunodeficiency virus.

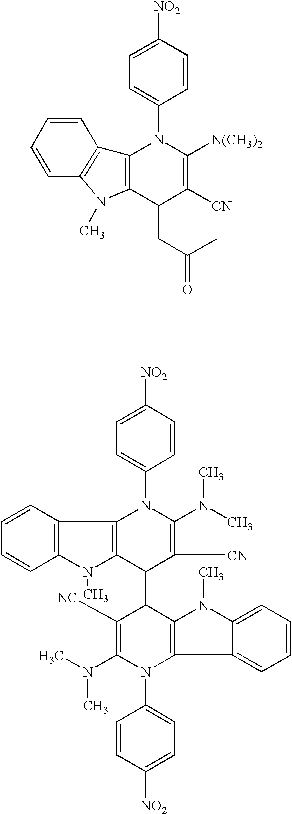

Compositions comprising effective amounts of such compounds also are described. These compounds and compositions can be used in a method for inhibiting the replication of retroviruses in a subject comprising administering an effective amount of the compound(s) or composition(s) comprising the compound to a subject to inhibit retroviral replication.

The present application claims priority from U.S. Provisional Application No. 60/256,556, filed on Dec. 18, 2000.

### FIELD

The present invention concerns benzoylalkylindolepyridinium compounds, pharmaceutical compositions comprising such compounds, and methods for making and using such compounds and compositions.

### BACKGROUND

Viruses cause a variety of human and animal illnesses. Many are relatively harmless and self-limiting, but the other end of the spectrum includes acute life-threatening illnesses such as hemorrhagic fever, and prolonged serious illnesses such as hepatitis B and acquired immune deficiency syndrome (AIDS). Unlike bacterial infections, where numerous suitable antibiotic drugs are usually available, there are relatively few effective antiviral treatments.

A. Viruses

Viruses consist of a nucleic acid surrounded by one or more proteins. A virus's nucleic acid typically comprises relatively few genes, embodied either as DNA or RNA. DNA genomes may be single or double-stranded (examples include hepatitis B virus and herpes virus). RNA genomes may be single strand sense (so-called positive-strand genomes; examples include poliovirus), single strand or segmented antisense (so-called negative-strand genomes; examples include HIV and influenza virus), or double-stranded segmented RNA genomes (examples include rotavirus, an acute intestinal virus).

Retroviruses represent a particular family of negative stranded RNA virus. The term “retrovirus” means that in the host cell the viral RNA genome is transcribed into DNA. Thus, information is not passing in the “normal” direction, from DNA to RNA to proteins, but rather in a “retrograde” direction, from RNA to DNA. To accomplish this change in direction, a retrovirus has one of a unique class of enzymes referred to as the reverse transcriptases. These enzymes are RNA-dependent DNA polymerases—that is, they synthesize DNA strands using the viral RNA genome as a template. Each species of retrovirus has its own reverse transcriptase. Once the reverse transcriptase copies the retroviral RNA genome, it uses its inherent DNA-dependent DNA polymerase activity—that is, the ability to synthesize DNA copied from other DNA—to generate a double-stranded DNA version of the viral DNA genome.

HIVs (human immunodeficiency viruses) are retroviruses of the lentivirus subfamily. The two known subfamily members that infect humans are called HIV-1 and HIV-2 (simian immunodeficiency virus, or SIV, is a closely related lentivirus that infects monkeys). Once the virus gains entry into the body, it attaches to human immune cells that express the CD4 receptor on their surface (CD4+ cells). CD4+ cells (which include “helper” and lymphocytes and monocytes), become the primary repository for the virus. HIV-1 isolates are categorized into two broad groups, group M and group 0. Group 0 comprises eight subtypes or clades, designated A through H.

B. Viral Therapeutics

Currently, only a limited number of drugs are approved for treating viral infections, such as human immune deficiency virus Type 1 (HIV-1) infection. Two broad families of anti-HIV drugs include the viral protease inhibitors, and the reverse transcriptase (RT) inhibitors. There are three main classes of RT inhibitors: (1) dideoxynucleoside (ddN) analogs, (2) acyclic nucleoside phosphonate (ANP) analogs, and (3) non-nucleoside reverse transcriptase inhibitors (NNRTIs).

The ddN and ANP nucleoside analog drugs are phosphorylated inside the cell. Once phosphorylated, they bind to the RT's substrate binding site. This is the site where the RT binds nucleotides (dATP, dCTP, dGTP, or dTTP, collectively referred to as dNTPs) so that they can be added to the growing DNA chain. When a nucleoside analog drug binds to the RT substrate binding site, it is integrated into the DNA, just as a normal dNTP would. But the enzyme cannot subsequently add dNTPs onto the incorporated nucleoside analog. Thus, the two classes of nucleoside analogs function as “chain terminators,” and thereby limit HW replication. These drugs have proven clinically effective against HIV infection, but resistance rapidly emerges due to mutations in and around the RT active site.

NNRTIs do not require phosphorylation or function as chain terminators, and do not bind at the substrate (dNTP) binding site. Known NNRTIs bind to a specific region outside the RT active site, and cause conformational changes in the enzyme that render it inactive. Known NNRTIs are highly potent and relatively non-toxic agents that are extremely selective for inhibition of HIV-1 RT. However, like the nucleoside analogs, their use is limited by the rapid emergence of resistant strains. In addition, they do not inhibit the RT activity of HIV-2, SIV and possibly some HIV-1 Group O isolates, nor do they prevent these viruses from replicating.

C. Pyrido-Indole Compounds

Ryabova et al. describe certain pyrido-indole compounds in “2-Formyl-3-Aryl-aminoindoles in the Synthesis of 1,2- and 1,4-Dehydro-5H-Pyrido-[3,2-b]-Indole (δ carboline) Derivatives,” Pharmaceutical Chemistry Journal, 30:579-583 (1996). For example, Ryabova et al. describe 1-(4-nitrophenyl)-2-dimethylamino-3-cyano4-(2-oxo-propyl)-5-methyl-1,4-dehydro-5H-pyrido [3,2-b]-indole (Compound 2).

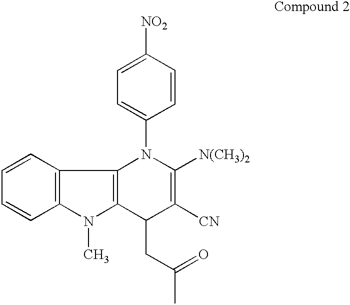

No biological data is provided for this compound.

D. Conclusion

The treatment of viral diseases, such as HIV disease, has been significantly advanced by the recognition that combining different drugs with specific activities against different biochemical functions of the virus can help reduce the rapid development of drug resistant viruses. However, even with combined treatments, multi-drug resistant strains of the virus have emerged. Therefore, there is a continuing need to develop new drugs, particularly antiviral drugs that act specifically at different steps of the viral infection and replication cycle.

### SUMMARY

The disclosed invention provides new antiviral compounds and pharmaceutical compositions comprising such compounds, particularly antiretroviral compounds and compositions, that address many of the problems noted above. These compounds, referred to as benzoylalkylindolepyridinum compounds (BAIPs), are effective against HIV isolates that have developed mutations rendering conventional drugs ineffective in their treatment. The BAIPs apparently do not require intracellular phosphorylation nor bind to the RT active site, which distinguishes their mechanism of action from the ddN and ANP nucleoside analog drugs. The BAIPs also may be distinguished from the NNRTIs, in part because the BAIPs bind to a different site on the RT enzyme. Moreover, unlike the NNRTIs, BAIPs of the present invention have been shown to be effective for limiting HIV-1, HIV-2, and SIV proliferation. Thus, BAIPs are broadly antiviral, non-nucleoside reverse transcriptase inhibitors (BANNRTIs).

Novel BAIPs have Formula I below.

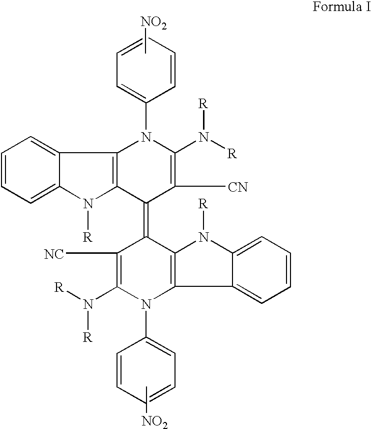

With reference to Formula I, R is selected from the group consisting of hydrogen and lower aliphatic, particularly lower alkyl, such as methyl. The nitro group (—NO2) can be at any ring position, i.e., ortho, meta orpara to the ring nitrogen, but typically is in the para position.

One novel compound of the present invention is shown below (Compound 2).

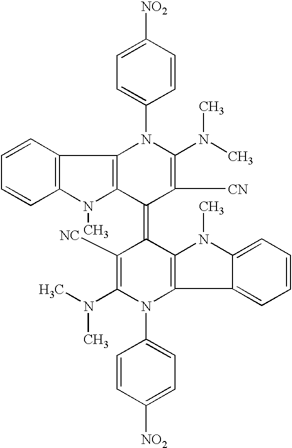

The present invention also provides a method for treating a subject, such as treating viral infections. The method comprises providing a compound having Formula II.

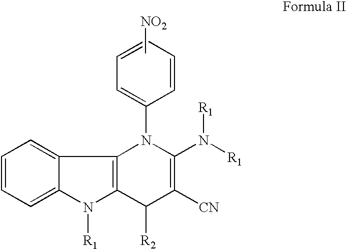

With reference to Formula II, R₁ is selected from the group consisting of hydrogen and lower aliphatic, particularly lower alkyl, such as methyl; and R₂ is selected from the group consisting of —CH₂COCH₃ and

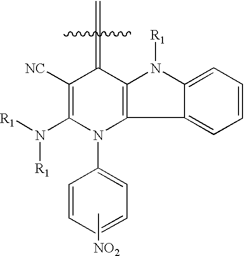

where R, is as stated for Formula II.

The compound is administered in effective amounts to subjects, such as a human or simian. A person of ordinary skill in the art will realize that the effective amount can vary. However, solely by way of guidance, an effective amount typically is from about 0.1 mg/kg body weight per day, to about 200 mg/kg body weight per day, in single or divided doses. The compound, or compounds, can be administered in any of a number of ways, including without limitation, topically, orally, intramuscularly, intranasally, subcutaneously, intraperitoneally, intravenously, or combinations thereof. The currently preferred administration method is intravenous. Such compounds also can be administered as pharmaceutical compositions, and hence may include other materials commonly found in pharmaceutical preparations, including other therapeutic agents.

The present invention also provides compositions comprising amounts of a compound or compounds effective to treat diseases, particularly viral infections. One likely mechanism of action is by inhibition of reverse transcriptase, and therefore effective amounts can be amounts sufficient to inhibit reverse transcriptase. Such compositions may further comprise inert carriers, excipients, diagnostics, direct compression binders, buffers, stabilizers, fillers, disintegrants, flavors, colors, lubricants, other active ingredients, other materials conventionally used in the formulation of pharmaceutical compositions, and mixtures thereof.

A method for treating a subject, particularly mammals, such as humans and simians, also is provided. The method first comprises providing a compound having Formula II, such as Compound 2, or a composition comprising Compound 2, as described above. An amount of the compound(s) or composition(s) effective to inhibit viral replication is then administered to a subject. The effective amount typically should be as high as the subject can tolerate. The currently preferred administration method is intravenous.

### BRIEF DESCRIPTION OF THE DRAWINGS

FIG. 1 is a graph of various concentrations of Compound 2 (μM) versus percent control which illustrates the effects of Compound 2 on virus particles released from infected cells, where virus associated p24 antigen (♦) was quantitated by antigen capture assay, RT activity (▪) was assessed by a homopolymeric(rA) template-primer system assay, and infectious units (▴) were quantitated by titration of cell-free supernatant on MAGI cells.

FIG. 2 is a photograph of Western blot gels with AIDS patient serum or with polyclonal antiserum to HIV-1 RT protein.

FIG. 3 is graph of concentration of Compound 2 versus percent control showing decreased (1) RT activity levels (·), which were quantitated in the cell-free supernatant from TNF-α stimulated ACH2 cells in the presence of Compound 2, and (2) infectious units (▪), which were quantitated in the cell-free supernatant from TNF-α stimulated ACH2 cells in the presence of Compound 2, (3) RT (∘) of a separate sample, and (4) infectious units (□) from a separate sample showing that under these conditions activities of RT and infectivity were recovered, where points on the graph represent means of triplicate tests from a representative experiment. RT activity levels also were measured in virus harvested from drug-free TNF-α stimulated ACH2 cells after treatment of those preparations with either freshly prepared Compound 2 or with a fluid phase in which the virus had been cleared by centrifigation from the Compound 2 treated cultures.

### DETAILED DESCRIPTION

I. Defintions

“Lower” as used herein refers to a compound or substituents having 10 or fewer carbon atoms in a chain, and includes all position, geometric and stereoisomers of such compounds or substituents.

“Aliphatic” refers to compounds having carbon and hydrogen molecules arranged in straight or branched chains including, without limiation, alkanes, alkenes and alkynes.

“Alkyl” as used herein refers generally to a monovalent hydrocarbon group formed by removing one hydrogen from an alkane. An alkyl group is designated generally as an “R” group, and has the general formula —CₙH₂ₙ₊₁.

II. Compounds

Novel compounds of the disclosed invention have Formula I.

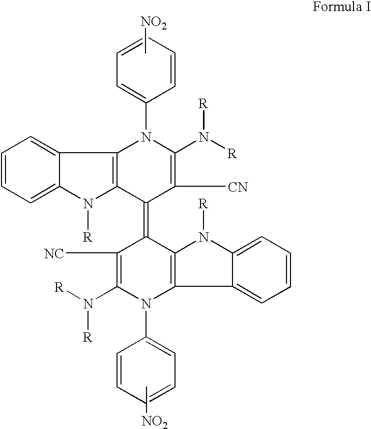

With reference to Formula I, R is selected from the group consisting of hydrogen and lower aliphatic, particularly lower alkyl, such as methyl. Compound 2 is an example of a compound having Formula I.

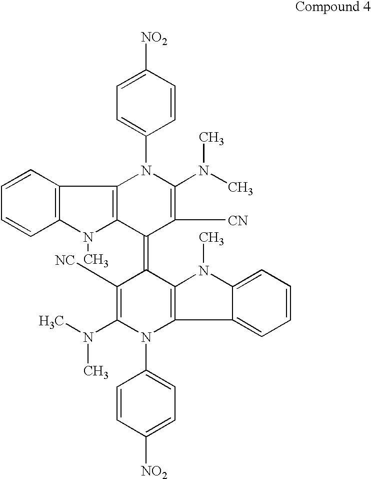

The present invention also is directed to a method of using compounds having Formula I and related biologically active compounds. These Formula I and related biologically active compounds have Formula II.

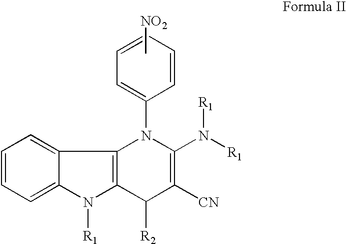

With reference to Formula II, R₁ is selected from the group consisting of hydrogen and lower aliphatic, particularly lower alkyl, such as methyl. R₂ is selected from the group consisting of —CH₂COCH₃ and

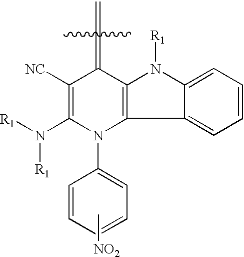

Examples of such compounds include biologically active Compounds 1 (above) and 2 (below).

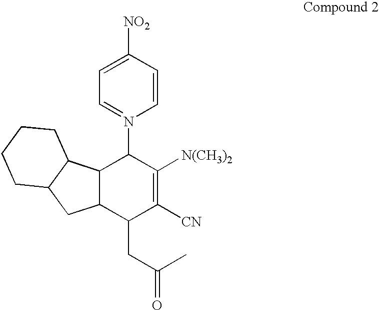

III. General Methods for Making BAIPs

Compound 2 can be made as described by Ryabova et al. in “2-Formyl-3-Aryl-aminoindoles in the Synthesis of 1,2- and 1,4-Dihydro-5H-Pyrido-[3,2-b]-Indole (δ Carboline) Derivatives,” Pharmaceutical Chemistry Journal, 30:579-583 (1996), which is incorporated herein by reference. Other methods also can be used to make such compound, as well as other compounds according to the present invention. Example 1 describes a method for making Compound 2 as well.

With reference to Scheme 1, in a first such method IV was deacylated by action of Et₃N in methanol to form 3-p-nitrophenylaminoindole V, yield 80%, m.p. 220-222° C. (MeOH), IR v/cm⁻¹: 3350, 1590; MS m/z 253 (M⁺). Formylation of V by treatment with Vilsmeier reagent produced the 2-formyl derivative VI, yield 96%, m.p. 237-238° C. (DMF—H₂O, 2:1); IR v/cm⁻¹: 3290, 1640, 1600, 1575; ¹H NMR ([²H₆]DMSO), §: 9.88 (1H, s, CHO), 11.85, 9.41 (2H, 2s, NH, NHC₆H₄NO₂), 7.48 (4H, A₂B₂ system, C₆H₄NO₂), 6.95-7.59 (4H, m, arom. protons); MS m/z281 (M⁺).

Condensation of aldehyde VI with the dinitrile of malonic acid (both in the presence of Et₃N at 20° C. or without Et₃N but under reflux) leads to dinitrile VII, yield 80% and 71%, respectively; a m.p.>270° C. (dioxane); IR, v/cm⁻¹: 3390, 3290, 2210, 1570; ¹H NMR ([²H₆]DMSO), 5: 8.19 (1H, s, CH), 11.17, 9.68 (2H, 2s, NH, NHC₆H₄NO₂), 7.52 (4H, A₂B₂ system, C₆H₄NO₂), 7.11, 7.67 (4H, m, arom. protons); MS m/z 329 (M⁺). Cyclization of dinitrile VII can occur in either of two directions: with participation of endo (indole) or exo (at position 3) cyclic NH groups.

Heating VII in DMF-MeOH (1:1) caused intramolecular cyclization to form VIII isolated as the semihydrate (Scheme 1) yield 60%, m.p. 280° C. (decomp., DMF-MeOH, 1:1).

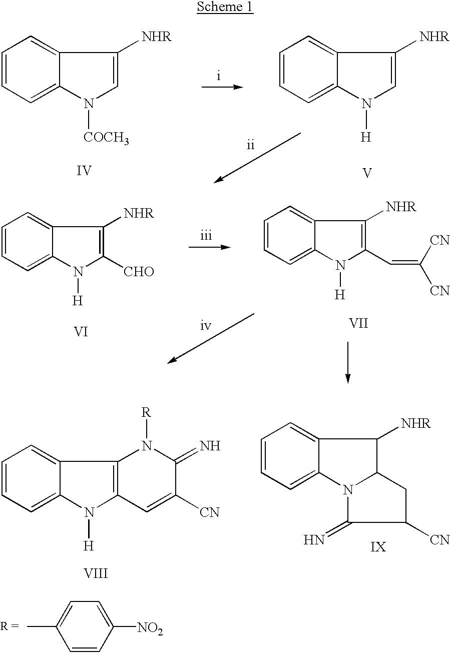

### Reagents and Conditions for Scheme 1:

i. Et₃N, MeOH, reflux two hours.

ii. POCL₃-DMF, 5-10° C., 0.25 hours; addition of a solution of 3 in DMF, standing of the mixture (20° C., 18 hours)

iii. PrOH, CH₂(CN)₂, reflux 5 hours, or PrOH, PrOH, CH₂(CN)₂, Et₃N, 5 hours, 20° C.

iv. DMF-MeOH, 1: 1, reflux 0.25 hours.

Spectroscopic Data for VII

IR v/cm⁻¹¹: 3320, 2200, 1620, 1600, 1580.

¹H NMR-DMSO-d₆, S: 6.17 (bs, 2H), 5.91 (d, 1H, H—C⁹), 6.74 (t, 1H, H—C⁸)

¹³C NMR ([²H₆]DMSO) §: 154.9 (C₂), 99.8 (C₃), 133.9 (C₄), 119.8 (C₄ₐ), 114.5 (C_{9b}), 139.9 (C₅ₐ), 128.8 (C₉ₐ), 113.1, 119.9, 126.2, 127.1 (C₆₉), 119.9, 131.1 (C_{2,3,5,6}), 148.1, 144.1 (C_{1,4}), 117.7 (CN).

MS m/z 329 (M⁺).

Scheme 2 shows an interesting and unexpected result that is obtained by methylating Compound VIII. Reacting VIII with methyl iodide in acetone in the presence of anhydrous K₂CO₃ adds the acetonyl anion to the molecule's 4 position, together with tris-alkylation. As a result, 1-nitrophenyl-2-dimethylamino-3-cyano-4-acetonyl-5-methyl-1,4-dihydropyrido[3,2-b]indole X is obtained, yield 75%, m.p. 198-199° C. (MeOH-dioxane, 3:1).

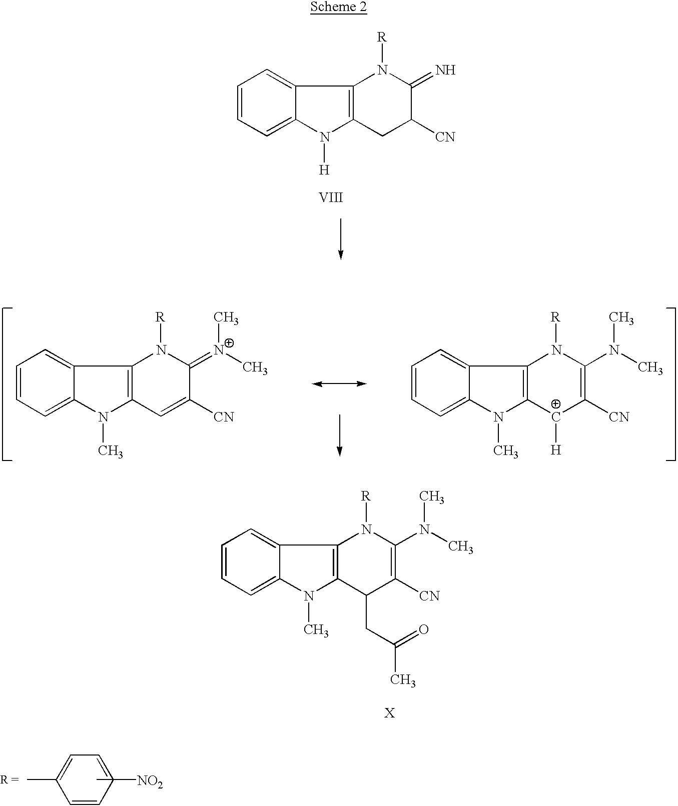

Reagent and Conditions:

MeI, acetone, anhydrous K₂CO₃, reflux 56-60 hours, MeI added to the reaction mixture every 7-8 hours.

### Spectroscopic Data for X

IR v/cm⁻¹: 1720, 2190.

¹H NMR ([²H₆]DMSO) d: 3.75 (3H, s, NMe-indole), 2.90 (6H, br.s, NMe₂), 2.10 (3H, s, CH₂COMe) 2.69 (2H, AB system Jₕₑₘ, 17 Hz, J¹_{vic} 9 Hz, J²_{vic} 5 Hz, CH₂COMe), 4.21 (1H, q, H—C₄), 7.89 (4H, A₂B₂ system, C₆H₄NO₂), 7.08-7.53 (4H, m, arom. protons). MS m/z 429 (M⁺), 372 (M⁺—CH₂COMe).

In Scheme 2, first tris-methylation appears to occur with formation of a positively charged species, and the acetonyl anion (formed in the reaction mixture in the presence of K₂CO₃) reacts at the electron-deficient position 4 to yield X. In the ¹H NMR spectrum of X (as distinct from VIII) a lower-field shift of the 9-H signal is not observed. The 1,4-dihydropyridine ring is not a flat system, and some data show that this ring has a boat conformation. Construction of molecular models for X, taking into account these data, shows that in this instance the p-nitrophenyl ring cannot influence the shape due to the anisotropic effect (as for VIII and so the signals for all the protons in the condensed benzene ring are within the same range (7.08-7.53).

Ryabova et al., Khim.-Farm. Zh., 30: 4245, (1996) reported the synthesis of 2-formyl-3-arylaminoindole derivatives by formylation of the corresponding 3-arylaminoindoles according to the Vilsmeier reaction. Despite the “enamine” character of VI, the aldehyde group in position 2 is still capable of entering the reaction typical of this moiety. For example, reactions with primary amines lead to the formation of Schiff bases and the interactions with compounds possessing an active methylene group yield 2-vinylindole derivatives. Reaction of VI with malononitrile formed the 2-dicyanovinyl-3-arylaminoindoles VII, which are used to synthesize new indoles and condensed indole derivatives.

Heating compound VII for a short time in acetone in the presence of potassium carbonate leads predominantly to the hydration of vinyl fragment with the formation of initial aldehyde VI. The δ-carboline cyclization dominates when VII is heated in a DMF-MeOH (1:1) mixture up to the boiling temperature, and VIII is obtained at a 73% yield. The δ-carboline structure of VIII was confirmed by ¹H NMR spectroscopic data (Ryabova et al., Pharm. Chem. J. 30: 579-584, 1996). The ¹H NMR spectrum of VIII in DMSO-d₆ includes the following signals (δ, ppm): 6.17 (bs, 2H), 5.91 (d, 1H, H—C⁹), 6.74 (t, 1H, H—C⁸), 7.23 (t, 1H, H—C⁷) and 7.42 (q, 1H, H—C⁶).²) 7.88 and 8.55 (A₂B₂ system, 4H, C₆H₄NO₂), 8.25 (s 1H, H—C⁴). A characteristic feature of the latter spectrum is a considerable upfield shift of the H—C⁹ proton signal (5.91 ppm) as compared to the signals of other protons of the benzene ring (6.74-7.42 ppm) and the analogous proton signals in the spectra of pyrrolo[1,2-a]indole (7.27-7.94 ppm) and 3-arylamino-2-formylindole X (6.95-7.59 ppm). Apparently, this shift of the H—C⁹ signal toward higher field strengths can be only due to the effect of anisotropic circular currents of the 4-nitrophenyl substituent in position 1, displaced out of the plane of the molecule as a result of steric constraints (the Dreiding molecular models). Thus, the experimental data confirmed the δ-carboline structure of VIII.

Alkylation of 3-aminoindole, initial aldehyde VI, 2-vinyl derivative VII, and 1,2-dihydro-δ-carboline VIII was used to develop a general method for making N-alkyl derivatives. This provided a common approach to obtaining compounds substituted at the exocyclic amino group and the nitrogen atom of the indole cycle. According to the mass-spectrometric data, methylation of VII by methyl iodide in acetone in the presence of potassium carbonate leads to the formation of a mixture of mono- and dimethyl derivatives, 2-formylindole, and 6-carboline X. Using column chromatography methods, aldehyde VI was isolated as was a bis-dimethyl derivative from this mixture. A side product in this reaction was 3-(4-nitrophenylamino)indole-2-carboxylic acid.

On heating in the presence of an aqueous alkali with dimethyl sulfate in acetone, compound VIII is methylated at the endo- and exocyclic nitrogen atoms (probably, via the stage of formation of the corresponding anion) yielding δ-carboline X from the reaction mixture (Scheme 3).

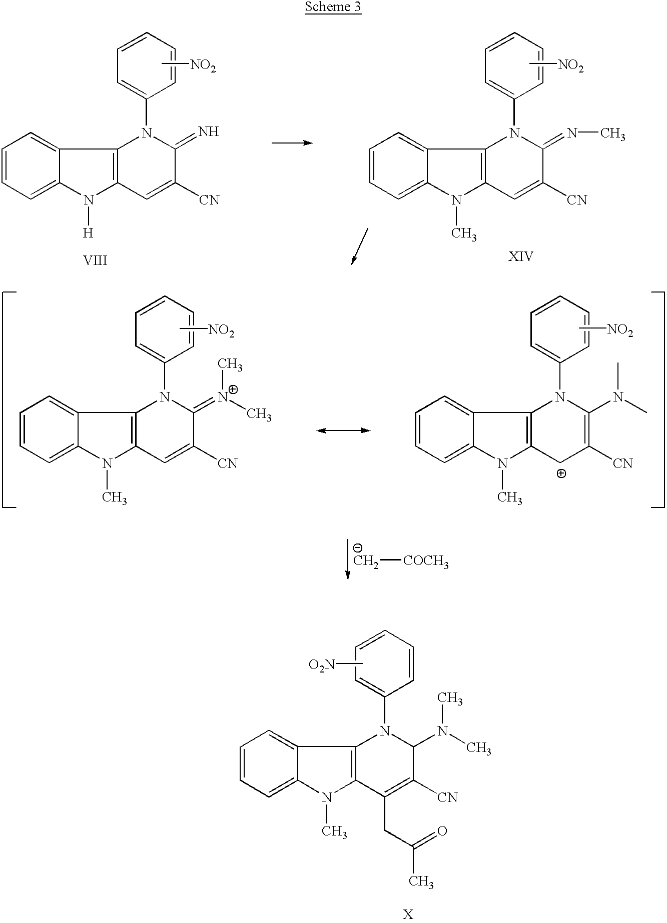

The ¹H NMR spectrum of X (Table 2) contains signals from two methyl groups: δ=3.18 ppm (s. 3H, 2-NMe) and 3.81 ppm (s, 3H. 5-NMe). On saturation of the low-field N-methyl group signal, the intensity of the doublet at 5=7.45 ppm increases by 8%, and that of the singlet at δ=8.50 increases by 14%. In contrast, saturation of the signal of the other methyl group leads to no increase in the intensity of signals from aromatic protons. At the same time, saturation of the low-field part (δ=−7.70 ppm) of the A₂B₂ system of signals from protons of the 4-nitrophenyl fragment increases by 4% the intensity of a doublet (δ=5.82 ppm) belonging to the proton at C⁹. The above NOE estimates unambiguously confirm the proposed structure of compound X, in which the methyl group at N⁵ approaches the positions of H—C⁴ and H—C⁶, while the proton at C⁹ is close to protons of the 4-nitrophenyl substituent in position 1. The comparatively small increase in intensity of the doublet due to C⁹ protons (δ=5.82 ppm), observed on saturation of the signal from ortho protons of the nitrophenyl fragment, is probably explained by increasing distance to this proton system as a result of displacement of the N¹-aryl substituent out of the molecular plane. This also leads to the upfield shift of the signal from H—C⁹.

A different reaction of VIII with methyl iodide is observed in the presence of potassium carbonate, whereby the final result is determined by the methylation medium. For example, prolonged heating of the components in acetone leads to trimethylation of the initial carboline, accompanied by attachment of the acetonyl anion in position 4. As a result, a tricyclic structure was obtained, in which the indole cycle is linked to the 1,4-dihydropyridine ring having a new functional substituent in position 4.

The dimethyl derivative X is apparently an intermediate involved in the formation of other compounds. This is confirmed by the fact that methylation of X using cyclohexanone or methylethylketone as solvents instead of acetone leads to 1-(4-nitrophenyl)₂-dimethylamino-3-cyano4-(2-oxocyclohexyl) and (3-oxo-2-butyl)-5-methyl-1,4-dihydro-δ-carbolines, respectively.

This initial stage may involve exhaustive methylation with the formation of a cation, in which the positive charge is delocalized between a dimethylamino group and position 4 of the molecule. It is this position to which the anion of a ketone (present in the reaction mass) is attached in the following stage with the formation of 1,4-dihydro-δ-carbolines.

The proposed structure of synthesized δ-carbolines was confirmed by spectroscopic data, primarily by the results of NMR measurements. For example, and with reference to compound X, the IR spectrum of this compound, measured as a Nujol mull, showed the absorption bands at 1720 cm⁻¹ (nonconjugated ketone) and 2190 cm⁻¹ (CN group); mass spectrum (m/z): 429 [M⁺], 372 [M⁺—CH₂COCH₃]; ¹H NMR spectrum in DMSO-d₆ (δ, ppm): 3.75 (s, 3H, NMe), 2.90 (bs, 6H, NMe), 2.10 (s, 3H, CH₂COCH₃), 2.69 (AB-system, 2H, Jₕₑₘ 17 Hz, J¹_{vic} 9 Hz, J²_{vic} 5 Hz CH₂COCH₃), 4.31 (q, 1H, H—C⁴), 7.89 (A₂B₂-system, 4H, C₆H₄NO₂), 7.08-7.53 (4H, aromatic protons).

The IR spectra of synthesized compounds were measured on a Perkin-Elmer Model 457 spectrophotometer using samples prepared as Nujol mulls. The mass spectra were obtained on a Varian MAT-112 mass spectrometer with direct introduction of samples into the ion source operated at an ionizing electron energy of 70 eV. The NMR spectra were recorded on a Varian XL-200 instrument (ISA) using TMS as the internal standard. The course of reactions was monitored and the samples were identified by thin-layer chromatography on Silufol UV-254 plates eluted in the chloroform methanol system (10:1). The data of elemental analyses coincided with the results of analytical calculations.

IV. Biological Activity

Compound 2 exerts broad anti-retroviral activity and has low cellular toxicity. Compound 2 initially was found active against HIV-1_{RF} in a standard screening cytoprotection assay (EC₅₀=0.1 μM and a CC₅₀>200 μM) that requires multiple rounds of viral infection. Range of action studies showed that Compound 2 also inhibited a panel of retroviruses, including laboratory and clinical isolates of HIV-1, HIV-1 isolates housing mutations that confer resistance to nucleoside and NNRTIs, monotropic and lymphotropic HIV-1 strains, as well as HIV-2 and SIV (Table 1).

TABLE 1
Antiviral Properties of Compound 2

| Virus | Cell | EC₅₀ | CC₅₀ | TI |
|---|---|---|---|---|
| HIV-1 RF | CEM-SS | 0.1 | >200 | >2000 |
| | | 0.078 | >200 | >2570 |
| HIV-1 IIIB | CEM-SS | 0.824 | >200 | >242 |
| | | 0.836 | 126 | 151 |
| HIV-1 OC/100 | CEM-SS | 4.68 | 116 | 24.9 |
| | | 1.19 | 113 | 94.5 |
| HIV-1 HEPT/236 | CEM-SS | 0.97 | 133 | 137 |
| HIV-1 CALO-R | CEM-SS | 1.10 | 122 | 110 |
| | | 1.14 | 176 | 153 |
| HIV-1 ddI-R | CEM-SS | 0.62 | 163 | 263 |
| HIV-1 DPS-R | CEM-SS | 0.5 | 123 | 247 |
| HIV-1 4X AZT | CEM-SS | 1.3 | 110 | 84.8 |
| HIV-1 A-17 | CEM-SS | 2.98 | 92.1 | 30.8 |
| | | 3.48 | 88.1 | 25.3 |
| HIV-1 6R/AZT | CEM-SS | 16.6 | 130 | 7.8 |
| | | 12.0 | 109 | 9.1 |
| HIV-1 6S/AZT | CEM-SS | 1.41 | 125 | |
| | | 0.5 | 68.7 | 136 |
| HIV-1 N119 | CEM-SS | 1.01 | 109 | 108 |
| | | 9.73 | 124 | 12.8 |
| HIV-2 ROD | CEM-SS | 2.64 | 162 | 61.1 |
| | | 4.79 | >200 | >41.7 |
| | | 0.37 | >200 | >539 |
| SIV | CEMx174 | 5.65 | >200 | >35.4 |
| | | 6.5 | 134 | 20.6 |

¹XTT antiviral assays were performed as described below in Example 4.
EC₅₀ values indicate the drug concentration that provided 50% cytoprotection.
CC₅₀ values reflect the drug concentrations that elicit 50% cell death. The XTT cytoprotection studies with HIV-1 were confirmed by measurement of supernatant RT, p24 and infectious virus titers.

Mechanistic studies showed no inhibitory activity of Compound 2 against RT when evaluated in vitro with recombinant p66/p51 RT using either the poly(rA) oligo(dT) or poly(rC) oligo(dG) template-primer systems. Likewise, Compound 2 did not affect virus binding or fusion to target cells, the activities of HIV-1 integrase or protease enzymes, or the nucleocapsid protein zinc fingers (Table 2).

TABLE 2
Mechanism of Action Studies with Compound 2

| Molecular Target¹ | Effect | |
|---|---|---|
| RT (rAdT and rCdG) | NI² | |
| Protease | NI | |
| Integrase | NI | |
| NCp7 Zn fingers | NI | |
| Biological Target | Effect | |
| Early Phase | | |
| HIV-1 Attachment | 40% reduction at 100 μM | |
| Time Course Assay | No inhibition of proviral DNA synthesis | |
| MAGI Assay | No reduction in blue cell formation at 200 μM | |
| Late Phase | | |
| ACH-2 Assay | 1) | No reduction of p24 |
| | 2) | 2) Virus protein processing normal (Western |
| | | blot) |
| | 3) | Particle morphology normal (EM) |
| | 4) | Reduction in RT activity in new virions |
| | 5) | Reduction of infectious title of new virions |

¹Attachment of HIV-1 to CEM-SS cells, binding of gp120 to CD4, and the effects of compounds on HIV-1 RT, PR and NCp7 were quantitated as described below in Examples 4 and 5.
²NI indicates that no inhibition was observed at the high test concentration (100 μM).

Thus, Compound 2 appeared not to act on any of the classical anti-HIV molecular targets.

The activity of Compound 2 was evaluated using a MAGI, cell-based, early-phase model of infection, described in Example 5. This assay requires virus binding, fusion, reverse transcription, integration of proviral DNA and the expression of Tat protein. Viruses were added to the MAGI cells in the presence or absence of Compound 2, and viral infectivity determined by scoring the number of blue foci. Compound 2 demonstrated no apparent inhibitory action. Since the agent had no effect on these early-phase events, the data suggested it acted during the late phase of infection, after the HIV provirus integrates into the host cell genome.

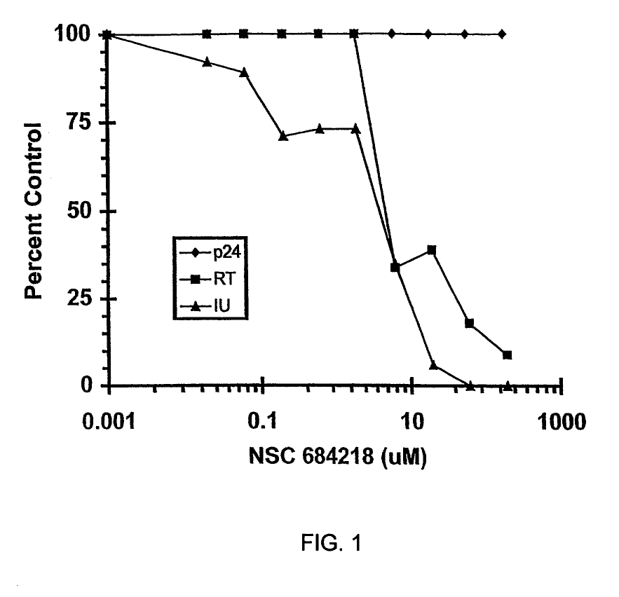

Compound 2 was evaluated in a late-phase model of HIV-1 replication, described in Example 7. This model uses ACH2 cells, which carry a latent HIV-1 infection. In this model, the ACH2 cells are treated with TNF-α which stimulates HIV-1 replication and virion production. Compound 2 had no effect on viral p24 antigen levels in the ACH2 cell culture supernatant, suggesting that virions were produced normally (FIG. 1). However, Compound 2 decreased virion-associated RT and viral infectivity levels in the culture supernatants in a concentration-dependent manner (FIG. 1). These observations were confirmed with latently infected U1 cells, chronically infected H9 cells, and other clones of latently infected ACH-2 cells under TNF-α induced or uninduced conditions (data not shown).

With reference to FIG. 1, ACH2 cells were stimulated with recombinant TNF-α in the absence or presence of various concentrations of Compound 2. Cell-free supernatants were collected and evaluated as described in Examples 4-6. Virus-associated p24 antigen (♦) was quantitated by antigen capture assay, RT activity (▪) was assessed by a homopolymeric(rA) template-primer system assay, and infectious units (▴) were quantitated by titration of the cell-free supernatant on MAGI cells wherein each blue cell represented an infectious unit. Examples 4-6. Each point represents the mean of triplicate cells from a representative experiment. Cell viability was unaffected at the relatively high test concentration of 200 μM, as assessed by XTT assay.

The MAGI and ACH2 data, taken together, show that Compound 2 acts during the late phase of infection, after the provirus has integrated into the host cell genome. In the ACH2 assay, a drug which acted intracellularly to inhibit HIV replication would reduce the amount of HIV released into the cellular supernatant. However, HIV virions apparently being produced in an essentially normal manner, since Compound 2 treatment did not reduce the amount of viral p24 antigen present in the culture supernatant. However, when the HIV virions were released from the cell into the culture media, they exhibited significant abnormalities. Compound 2-treated cells showed reduced virion-associated RT activity and viral infectivity levels, and the degree to which the activity was reduced was directly related to the concentration of Compound 2.

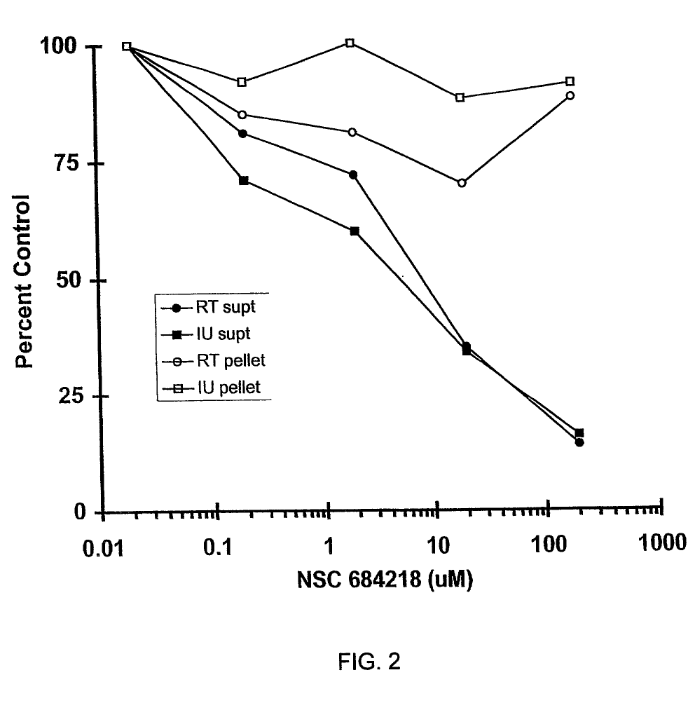

To further investigate the observed abnormalities, the HIV-1 virions released from Compound 2-treated cells were compared to control in Western blot and protein analysis and electron microscopy. TNF-α stimulated ACH2 cells were treated with either Compound 2 or control solution, and cell-free supernatants were centrifuged to pellet the virus particles. Samples were subjected to Western blot analysis with AIDS patient serum or with polyclonal antiserum to HIV-1 RT protein as shown by FIG. 2. The positions of gp120, Pr55^{gag} precursor polypeptide, p24 capsid (CA) protein, p17 matrix (MA) protein, integrase (IN), the p66 subunit of HIV-1 RT and p51 subunit of HIV-1 RT are indicated in FIG. 2. This analysis revealed a normal complement of fully mature (processed) HIV-1 proteins, including both subunits of the RT protein, in both control and Compound 2-treated supernatant. Electron micrographs of virus particles were obtained to assess morphological changes in virus particles treated with compounds of the present invention. Electron microscopy revealed no morphologic differences between virions obtained from control and Compound 2-treated cells. Thus, although virions released from Compound 2-treated cells had lower RT activity and were less infectious than virions released from control-treated cells, there were no abnormalities in virion morphology or protein composition that explained the difference.

### A. Compound 2 is a Prodrug

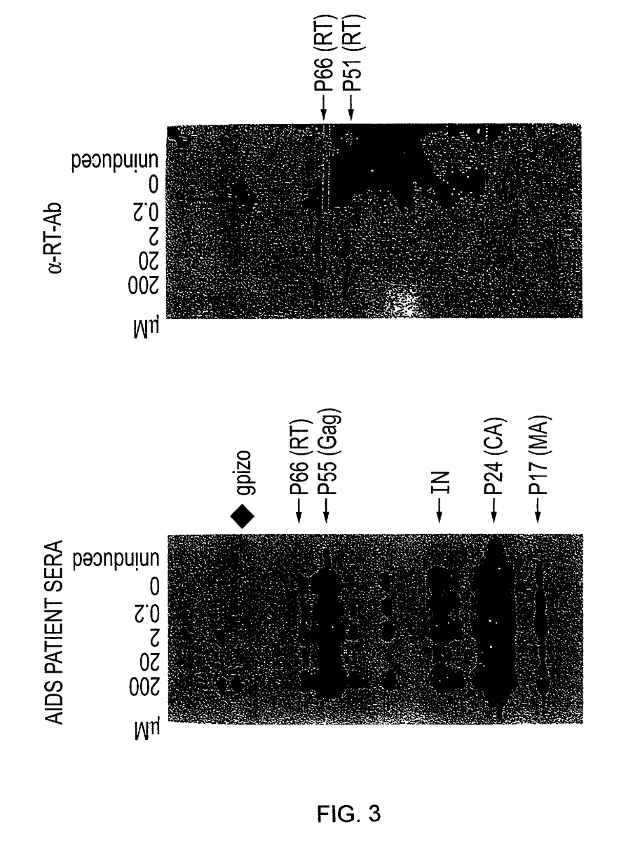

The actual mechanism of action of Compound 2 became apparent partially from studies in which virion-associated RT levels were measured following centrifugation of virus particles in the virus-rich ACH-2 culture media. With reference to FIG. 3, RT activity (·) and infectious units (▪) were quantified in the cell-free supernatant from TNF-α stimulated ACH2 cells in the presence of Compound 2. Activity levels decreased as the concentration of Compound 2 increased. A separate set of samples was centrifuged and the fluid phase removed prior to quantifying RT levels (∘) and infectious units (□) of the virus pellet. Removing the culture fluid from the centrifuged virus particles allowed recovery of RT activities and virus infectivity at levels equivalent to those found in virions from untreated ACH-2 cultures (FIG. 3). This indicated that Compound 2 was a prodrug that had been converted into an active and reversible RT inhibitor during the 72-hour culture period. This was confirmed by a study in which the RT activity in a lysate of normal HIV-1 virions was inhibited by addition of virus-depleted culture supernatant from drug-treated ACH-2 cells. In contrast, addition of drug-free culture media or fresh drug to the normal virions did not inhibit their RT activity.

VI. Summary

Compounds 2 and 4 are novel RT inhibitors with truly broad-spectrum activity against retroviral RT enzymes and against infection by a broad range of retroviruses, including HIV-1, HIV-2 and SIV. BAIPs demonstrated antiviral activity against laboratory isolates of HIV-1 and a panel of clade-representative clinical isolates in PBMC cultures at submicromolar levels. More impressive though was the ability of the BAIPs to inhibit the replication of a panel of HIV-1 variants carrying mutations in RT that confer resistance to AZT and various NNRTIs such as oxithiin carboxanilide (L-100→I), thaizolobenzimidazole (V-108→I), calanolode (T-139→I), diphenylsulfone (Y-181→I), 3TC (M-184→I) and others. The ability of the BAIPs to inhibit the enzymatic RT activities and replication of this wide array of retroviruses distinguished it from classical NNRTI type molecules that are HIV-1 specific and can be typically rendered ineffective by one or more single mutations in the HIV-1 RT enzyme. Thus, the BAIPs truly represent the first reported example of a broadly antiretroviral NNRTI (BANNRTI).

The BAIPs have been found to inhibit not only all strains of HIV-1 tested, but also the replication of HIV-2 and SIV. This property sets the BAIPs apart from other NNRTI-type agents. The BAIPs may be used for therapy to individuals already carrying HIV-1 variants that are resistant to AZT or classical NNRTI molecules.

Classical NNRTIs bind noncovalently to the non-substrate binding site of the RT enzyme, and mutations in this region of the enzyme result in loss of sensitivity to the agents. Likewise, nucleoside analogs interact with RT in the substrate binding pocket, and mutations in this region of the enzyme result in resistance to the respective nucleoside analogs. Because BAIPs exert such distinct antiviral properties from the classical NNRTIs and have such a different structure from nucleoside analogs, BAIPs likely interact with RT in a different manner that classical NNRTIs. A series of computational studies were performed that predict the most likely binding site for BAIPs. Such studies suggested that BAIPs bound tightly in a previously unidentified pocket near the Asp triad in the active site of the RT enzyme. Together, these studies set the BAIP molecules apart as a new class of RT inhibitors, the BANNRTIs.

VI. Pharmaceutical Compositions Comprising Compounds 1 and 2

The vehicle in which disclosed compounds can be delivered include pharmaceutically acceptable compositions of the drugs. Any of the common carriers, such as sterile saline or glucose solution, can be used with the compounds provided by the invention. Routes of administration include, but are not limited to, oral and parenteral routes, such as intravenous (iv), intraperitoneal (ip), rectal, topical, ophthalmic, nasal, transdermal, and combinations thereof.

The drugs may be administered intravenously in any conventional medium for intravenous injection, such as an aqueous saline medium, or in blood plasma medium. The medium also may contain conventional pharmaceutical adjunct materials such as, for example, pharmaceutically acceptable salts to adjust the osmotic pressure, lipid carriers such as cyclodextrins, proteins such as serum albumin, hydrophilic agents such as methyl cellulose, detergents, buffers, preservatives and the like. A more complete explanation of parenteral pharmaceutical carriers can be found in Remington: The Science and Practice of Pharmacy (19^{th} Edition, 1995) in chapter 95. The compositions are preferably in the form of a unit dose in solid, semi-solid and liquid dosage forms such as tablets, pills, powders, liquid solutions or suspensions.

VII. Administering Compounds

The present invention provides a treatment for HIV and SIV disease, perhaps by RT inhibition, and associated diseases, in a subject such as an animal, for example a monkey or human. The method includes administering a compound, or compounds, of the present invention, or a combination of the compound or compounds and one or more other pharmaceutical agents. The compound, or compounds, can be administered to the subject in a pharmaceutically compatible carrier. The compound, or compounds, are administered in amounts effective to inhibit the development or progression of HIV and SIV disease. Although the treatment can be used prophylactically in any patient at significant risk for such diseases, subjects can also be selected using more specific criteria, such as a definitive diagnosis of the condition.

The disclosed compounds are ideally administered as soon as possible after potential or actual exposure to viral infection. For example, once viral infection has been confirmed by laboratory tests, a therapeutically effective amount of the drug is administered. The dose can be given by frequent bolus administration.

Therapeutically effective doses of the compounds of the present invention can be determined by one of ordinary skill in the art. For example, effective doses can be such as to achieve tissue concentrations that are at least as high as the EC₅₀. The low cytotoxicity of the BAIP makes it possible to administer high doses, for example 100 mg/kg, although doses of 10 mg/kg, 20 mg/kg, 30 mg/kg or more are contemplated. Thus, the dosage range likely is from about 0.1 to about 200 mg/kg body weight orally in single or divided doses, more likely from about 1.0 to 100 mg/kg body weight orally in single or divided doses. For oral administration, the compositions are, for example, provided in the form of a tablet containing from about 1.0 to about 1000 mg of the active ingredient. Symptomatic adjustment of the dosage to the subject being treated can be achieved by suing tablets of varying amounts of compound, such as 1, 5, 10, 15, 20, 25, 50, 100, 200, 400, 500, 600, and 1000 mgs of the active ingredient.

The specific dose level and frequency of dosage for any particular subject may be varied and will depend upon a variety of factors as will be known to a person of ordinary skill in the art. These include the activity of the specific compound, the metabolic stability and length of action of that compound, the age, body weight, general health, sex, diet, mode and time of administration, rate of excretion, drug combination, and severity of the condition of the host undergoing therapy.

The pharmaceutical compositions can be used in the treatment of a variety of retroviral diseases caused by infection with retroviruses that require reverse transcriptase activity for infection and viral replication. Examples of such diseases include HIV-1, HIV-2, and the simian immunodeficiency virus (SIV).

The present invention also includes combinations of a BAIP compound, or BAIPs, of the present invention with one or more agents useful in the treatment of viral diseases, such as HIV disease. For example, the compounds of this invention may be administered, whether before or after exposure to the virus, in combination with effective doses of other antivirals, immunomodulators, anti-infectives, or vaccines. The term “administration” refers to both concurrent and sequential administration of the active agents.

Examples of antivirals that can be used in combination with the BAIP RT inhibitors of the invention are: AL-721 (from Ethigen of Los Angeles, Calif.), recombinant human interferon beta (from Triton Biosciences of Alameda, Calif.), Acemannan (from Carrington Labs of Irving, Tex.), ganciclovir (from Syntex of Palo Alto, Calif.), didehydrodeoxythymidine or d4T (from Bristol-Myers-Squibb), EL10 (from Elan Corp. of Gainesville, Ga.), dideoxycytidine or ddC (from Hoffman-LaRoche), Novapren (from Novaferon labs, Inc. of Akron, Ohio), zidovudine or AZT (from Burroughs Wellcome), didanosine, lamiduvine, delavirdine, nevirapine, ribavirin (from Viratek of Costa Mesa, Calif.), alpha interferon and acyclovir (from Burroughs Wellcome), indinavir (from Merck & Co.), 3TC (from Glaxo Wellcome), Ritonavir (from Abbott), Saquinavir (from Hoffmann-LaRoche), nelfinavir, and others.

Examples of immunomodulators that can be used in combination with the BAIPs of the invention are AS-101 (Wyeth-Ayerst Labs.), bropirimine (Upjohn), gamma interferon (Genentech), GM-CSF (Genetics Institute), IL-2 (Cetus or Hoffman-LaRoche), human immune globulin (Cutter Biological), IMREG (from Imreg of New Orleans, La.), SK&F106528, and TNF (Genentech).

Examples of some anti-infectives with which the BAIPs can be used include clindamycin with primaquine (from Upjohn, for the treatment of pneumocystis pneumonia), fluconazlone (from Pfizer for the treatment of cryptococcal meningitis or candidiasis), nystatin, pentamidine, trimethaprim-sulfamethoxazole, and many others.

The combination therapies are not limited to the lists provided, but include any composition for the treatment of HIV disease and related retroviral diseases (including treatment of AIDS).

### VI. EXAMPLES

The following examples are provided to exemplify certain particular features of working embodiments of the present invention. The scope of the present invention should not be limited to those features exemplified.

### Example 1

This example describes methods for making Compound 2 and related compounds.

2-Cyano-3-[3-(4-nitrophenylamino)-2-indolyl]acrylic acid nitrile (VII, Scheme 1).

Method 1. A mixture of 3.65 g (13 mmole) of compound VI, 1.6 g (24 mmole) malononitrile, 0.25 ml (2 mmole) triethylamine, and 73 ml of 2-propanol was stirred for 5 h at 20° C. and allowed to stand at this temperature for 16 h. The precipitate was separated by filtration and washed with 2-propanol to obtain 3.3 g of VII.

Method 2. A mixture of 3 g (11 mmole) of Compound VI, 1.5 g (22 mmole) malononitrile, and 60 ml of 2-propanol was refluxed for 4 h and allowed to stand for 16 h at 20° C. Then the reaction mixture was treated as in method 1 to obtain 2.7 g of VII.

Method 3. A suspension of 0.3 g (1 mmole) of N-acetylated derivative of VI, 0.1 g (1.5 mmole) malononitrile, and 0.13 g (1.5 mmole) fused sodium acetate in 5 ml of acetic acid was stirred for 0.5 h at 20° C., followed by 3 h at 80° C. Then 0.1 g of malononitrile was added and the mixture was stirred for another 5 h at 20° C. Then the mixture was cooled, and the precipitate was separated by filtering and washed with AcOH, water, and MeOH to obtain 0.05 g of VII.

1-(4-Nitrophenyl)-2-imino-3-cyano-1,2-dihydro-5H-pyrido [3,2-b]-indole (VIII, Scheme 1).

Method 1. A mixture of 3.3 g (10 mmole) of nitrile VII, 15 ml MeOH, and 15 ml DMF was heated to boiling. As a result, VII dissolved and a new precipitate appeared. This suspension was refluxed for 5 min and cooled. The precipitate was separated by filtering and washed with MeOH to obtain 2.4 g of VIII. ¹³C NMR spectrum in DMSO-d₆ (δ, ppm): 154.9 (C²), 99.8 (C³), 133.9 (C⁴), II, 114.5 (C^{9b}). 139.9 (C₅ₐ), 128.8 (C^{9b}), 113.1, 119.9. 126.2, 127.1 (C⁶—C⁹), 119.9, 131.1 (C^{2′}, C^{3′}, C^{5′}, C^{6′}′,) 148.1, 144.1 (C^{1′ C}⁴′), 117.7 (CN).

Method 2. A mixture of 0.33 g (1 mmole) of nitrile VII and 0.4 g (3 mmole) of calcined potassium carbonate in 10 ml of acetone was refluxed for 15 min. The precipitate was separated by filtering and washed with water to obtain 0.05 g of VII. The acetone mother liquor was evaporated, and the residue triturated with diethyl ether to obtain 0.17 g (61%) of VIII.

Methylation of 3-(4-nitrophenylamino)indole. To a mixture of 1.3 g (5 mmole) of 3-(4-nitrophenylamino)indole, 16 ml DMF, and 2.1 g (15 mmole) of calcined potassium carbonate was added 5 ml MeI and the mixture was stirred at 80° C. for 60 h, with 2 ml MeI added each 6 h (to a total of 20 ml). The mixture was cooled, the remainding potash separated by filtering and washed with DMF, and the filtrate was evaporated. The residue was triturated with diethyl ether on adding a minimum amount of MeOH and filtered. The filtrate was evaporated, and the residue chromatographed on a silica get column with chloroform. Five sequential 100 ml fractions were collected, and the third and fifth fractions containing individual products were evaporated. Fraction 1 yielded 0.6 g (42%) of 1-methyl-3-[N-methyl-N-(4-nitrophenyl)amino]indole, and fraction 3 yielded 0.4 g of 3-[N-methyl-N-(4-nitrophenyl)amino]indole.

1-(4-Nitrophenyl)-2-methylimino-3-cyano-5-methyl-1,2-dihydro-5H-pyrido[3,2-b]indole (XIV, Scheme 3). To a solution of 2 g (50 mmole) of NaOH in 2 ml water was added 100 ml acetone and 3.3 g (10 mmole) of VIII, and the mixture was heated to boiling on stirring and refluxed for 5 min. To this mixture was added 4 ml (40 mmole) of Me₂SO₄ and the boiling was continued with stirring for 6 h. Another 4 ml of Me₂SO₄ was added and the mixture was refluxed for another 6 h. Then the mixture was cooled, the precipitate separated by filtration, washed with acetone, and dissolved in 500 ml of boiling water. The solution was filtered hot, cooled and alkalified with 1N KOH (15 ml). The precipitate was filtered and washed sequentially with water, 2-propanol, and diethyl ether to obtain 2.1 g of XIV.

1-(4-Nitrophenyl)-2-dimethylamino-3-cyano-4-(2-oxo-propyl)-5-methyl-1,4-dihydro-5H-pyrido-[3,2-b]indole (XI, Scheme 3)

Method 1. To a suspension of 2.15 g (6.5 mmole) of VIII and 3.6 g (26 mmole) of calcined potassium carbonate in 80 ml of acetone was added 2 ml MeI and the mixture was refluxed on stirring for 60 h, with 2 ml MeI added each 7-8 h. Then the mixture was cooled and the remaining potash separated by filtering and washed with acetone. The filtrate was evaporated, and the residue triturated with water, filtered, and washed with water and methanol to obtain 2.1 g of a technical-purity product 1-(4-nitrophenyl)₂-dimethylamino-3-cyano-4-(oxo-propy)-5-methyl-1,4-dihydro-5H-pyrido[3,2-b]indole. The product was purified by boiling with 20 ml MeOH, after which the insoluble precipitate was filtered to obtain 1.5 g of Compound 2-(4-nitrophenyl)₂-dimethylamino-3-cyano4-(oxo-propyl)-5-methyl-1,4-dihydro-5H-pyrido[3,2-b]indole.

Method 2. A mixture of 1.07 g (3 mmole) of XIV, 0.83 g (6 mmole) calcined potassium carbonate, 70 ml acetone, and 2 ml MeI was refluxed with stirring for 45 h, followed by a procedure similar to that in method 1. This yielded 0.85 g of X, which was identical to the product obtained by method 1.

1-(4Nitrophenyl)-2-dimethylamino-3-cyano-4-(2-oxo-2-butyl)-5-methyl-1,4-dihydro-5H-pyrido-[3,2-b]indole. To a suspension of 0.33 g (1 mmole) of VIII and 0.65 g (4.7 mmole) of calcined potassium carbonate in 20 ml of methylethylketone was added 2 ml MeI. The mixture was refluxed with stirring for 41 h, with 2 ml MeI added each 6 h. The mixture was cooled and the remaining potash separated by filtering and washed with diethyl ether, water, and methanol. The residue was mixed with chloroform and the solution filtered and evaporated. The residue was triturated with ether, and the precipitate was filtered and washed with ether to obtain 0.1 g of 1-(4-nitrophenyl)-2-dimethylamino-3-cyano4-(2-oxo-2-butyl)-5-methyl-1,4-dihydro-5H-pyrido-[3,2-b]indole.

### Example 2

This example describes virus replication inhibition assays that have been performed. The established human cell lines and laboratory-derived virus isolates (including drug resistant virus isolates) used in these evaluations have previously been described (Weislow et al., 1989; Rice and Bader, 1995). The antiviral activities and toxicity profiles of the compounds were evaluated with CEM-SS cells and HIV-1_{RF} using the XTT (2,3-bis[2-methoxy4-nitro-5-sulfophenyl]-5-[(phenylamino)carbonyl]-2H-tetrazolium hydroxide) cytoprotection microliter assay which quantifies the ability of a compound to inhibit virus-induced cell killing or to reduce cell viability itself (Weislow et al., 1989; Rice and Bader, 1995). The data are reported as the concentration of drug required to inhibit 50% of virus-induced cell killing (EC₅₀) and the concentration of drug required to reduce cell viability by 50% (CC₅₀). HIV-1 isolates utilized included common laboratory strains (RF, IIIB and MN), as well as a panel of HIV-1 clinical isolates (Rice et al., 1997). The pyridinone-resistant HIV-1_{A17} isolate was obtained from Emilio Emini at Merck Sharpe and Dohme Laboratories. CEM, U1, ACH-2, HeLa-CD4-LTR-β-gal, 174×CEM, and H9/HTLV-IIB NIH 1983 cell lines were obtained from the AIDS Research and Reference Reagent Program (National Institute of Allergy and Infectious Disease, National Institutes of Health, Bethesda, Md.), as were the HIV-2ROD and the SIV isolates. Phytohemagglutinin-stimulated human peripheral blood lymphocytes and monocyte/macrophages were prepared and utilized in antiviral assays as previously described (Rice et al., 1996), and levels of virion-associated p24 in cell-free culture supernatants were determined via antigen capture ELISA (Beckman Coulter).

### Example 3

This example describes integrase, protease, RT and NC zinc finger assays that have been performed. In vitro inhibitory activity against recombinant HIV-1 protease was performed with a reverse-phase high-pressure liquid chromatography assay utilizing the Ala-Ser-Glu-Asn-Tyr-Pro-Ile-Val-Glu-amide substrate (multiple Peptide System, San Diego, Calif.) (Rice et al., 1993a). The in vitro actions of compounds on 3′-processing and strand transfer activities of recombinant HIV-1 integrase were assayed according to Bushman and Craigie (1991), but with modifications (Turpin et al., 1998). The action of compounds on the RNA-dependent polymerase activity of recombinant HIV-1 p66/p51 RT was determined by measuring incorporation of [³²P]TTP or [³²P]GTP into the poly rA:oligo dT(rAdT) or poly rC:oligo dG(rCdG) homopolymer template-primer systems, respectively, while the inhibition of drug on the DNA-dependent polymerase activity of purified recombinant HIV-1 RT was determined by measurement of incorporation of [³²P]TTP or [³²P]GTP into the polydA:oligodT)dAdT) or polydC:oligodG(dCdG) homopolymer template-primer systems, respectively (Pharmacia Biotech, Piscataway, N.J.). Reactions were performed in the presence or absence of the drug as described previously (Rice et al., 1997). Reactions were terminated with ice-cold 10% trichloroacetate, filtered through GF/C filter under vacuum, and the filters were then washed with 100% ethanol and [³²P] incorporation quantitated by Cerenkov counter. The LTR region of the HIV-1 gemonic RNA was prepared from a pGEM LTR by in vitro transcription with T7 RNA polymerase (Promega, Madison, Wis.). In pGEM LTR, LTR region from pNL⁴-3 was inserted into the polyliker of pGEM (Promega) in the orientation that the sense LTR RNA were made when T7 RNA polymerase was used. The rest of steps for the preparation of heteropolymeric primer-template and RT reaction was performed as described (Gu et al., 1993).

Virion-associated RT activity was performed as described previously (REF) in the presence or absence of compound with the homopolymeric template-primer (rAdT, rCdG, dAdT and dCdG) (Pharmacia Biotech, Piscataway, N.J.) or heteropolymeric template-primer prepared as described above. HIV-2_{ROD10} and SUV virions were obtained by transfection of proviral DNA into HeLa cells.

### Example 4

This example describes RNase H cleavage assays that have been performed. An α-[³²P]-uridine-labeled RNA template (81 nucleotides in length) was hybridized to a 20-base DNA oligonucleotide in the presence of 50 mM Tris-HCl, pH 8.0, 50 mM NaCl, 2.0 mM dithiothreitol, 100 μg/ml acetylated bovine serum albumin, and 10 mM CHAPS as previously described (Gao et al., 1998). For these reactions, 100 ng of RNA (approximately 50,000 cpm) and 20 ng ofDNA (oligonucleotide 3352, 5′TTCTCGACCCTTCCAGTCCC 3′) were utilized. Purified HIV-1 wild type RT (45 ng) was mixed with COMPOUND 4 such that the final concentrations were 0.1, 1.0, 10 or 100 μM, and the reactions were initiated by the addition of 60 mM MGCI₂ and the annealed RNA/DNA complex in a final volume of 12 l. This mixture was incubated at 37° C. for 1 minute with Compound 4 or for various times without the compound. Reactions were terminated by the addition of 2× loading buffer, and the products were heat denatured and resolved on a 15% denaturing polyacrylamide-7M Urea gel in TBE buffer at 1600 Volts for approximately 90 minutes. Gels were dried and exposed for autoradiography overnight, and the film was developed with a Kodak RP X—OMAT processor.

### Example 5

This example describes MAGI cell assays that have been performed. The MAGI cell indicator line was obtained from the AIDS Research and Reference Program, Division of AIDS, National Institute of Allergy and Infectious Disease. MAGI cells are a HeLa cell line that both expresses high levels of CD4 and contains a single integrated copy of a beta-galactosidase gene under the control of a truncated human immunodeficiency virus type 1 (HIV-1) long terminal repeat (LTR). These cells maintained in DMEM medium supplemented with 5% fetal bovine serum (FBS), 100U of penicillin G sodium, 0.1 mg of streptomycin sulfate, 0.2 mg G418 sulfate, and 0.1 mg of hygromycin B per ml.

MAGI cells and an HIV-1 env- and Tat-expressing HeLa (HL2/3) cell line were used to perform a fusion assay. Tat activates gene expression from the HIV LTR, and therefore upon fusion of MAG1 and HL2/3 cells, tat expressed in HL2/3 cells (Ciminale et al., 1990) would activate β-galactosidase expression in MAGI cells. MAG1 or HL2/3 cells (2.5×10⁵ in 500 μl 5% FBS/DMEM) were preincubated with the tested compound for 1 hour at 37° C., respectively. At the end of preincubation, two cell lines were mixed at 1:1 ratio and were continued incubated for another 16 hours. The cells were then fixed and stained for the expression of β-galactosidase with indolyl-β-D-galatopyranoside (X-Gal) as described previously (Kimpton and Emerman, 1992). The numbers of blue cells were counted by light microscopy.

MAGI cells were also used to examine the effects of compounds on virus replication, from attachment through early gene expression. In these assays, the LTR-driven β-galactosidase gene in MAGI cells would not be activated until the incoming virus had penetrated the cell, reverse transcribed its RNA genome, generated the double-stranded proviral DNA, integrated the proviral DNA into the host cell genome, and expressed its tat gene. The assay was preformed as previously described with modifications (Howard et al., 1998). The virus stock used in the assay was prepared either from TNF-α-induced U1 cells (HIV_{IIIB}) or pNL4-3-transfected from HeLa cells transfected with the pNL4-3 plasmid containing HIV-1 proviral DNA. Viruses were diluted in 200 μl DMEM medium supplemented with 5% fetal bovine serum (FBS), and were titrated to generate approximately 300 blue cells per well in 24 well plates. Viruses were added to the MAGI cells in the presence or absence of the test compound. After 2 hours incubation at 37° C., the virus was removed, the cells were washed and 1 ml 5% FBS/DMEM medium with or without the test compound was added to the cells. For the time-of-addition assay, the compound was added at time zero when the infection was initiated, or at 2, 4, 8 or 24 hours post initiation of the infection. For the time-of-removal assay, the compound was added to all wells at the beginning of infection and was then removed at 2, 4, 8, 24 or 48 hours thereafter. The cells were washed once with medium after removal of the drug followed by the readdition of 1 ml 5% FBS/DMEM fresh medium. Forty-eight hours post initiation of infection, cells were fixed and stained as described above.

To titrate the infectivity of viruses harvested from the drug-treated chronic infected cells, MAGI cells were also used. Either 500 μl total culture media or 200 μl pelleted viruses were added to the 24 well culture plates in the presence 20 μg/ml DEAE-dextran for 3 hours at 37° C. prior to the addition of 2 ml of media. The cultures were fixed and stained as described above.

### Example 6

This example describes PCR analysis of nascent proviral DNA. MAGI cells were plated at a density of 4×10⁵/well in a 6-well plate. Twenty-four hours later, the cells were infected with HIV_{IIB} viruses in 500 μl 5% FBS/DMEM in the presence or absence of the compound. HIV_{IIB} viruses were prepared from TNF-α-induced U1 cells and the amount used in one infection was titrated as the amount producing 1000 blue colonies. Four hours post-infection, the cells were trypsinized, washed and digested at 55° C. for 1 hour with 100 μg/ml protease K in 100 μl buffer containing 0.5% Triton X-100, 100 mM NaCl, 50 mM Tris (pH 7.4), and 1 mM EDTA. To inactivate protease K, the samples were then heated at 100° C. for 15 minutes. PCR reactions were performed using M661 and M667 primers (Zack et al., 1990) and 5 μl sample was used in each reaction.

### Example 7

This example describes ACH2 latently-infected cell assays that have been performed. ACH2 cells were maintained in RPMI 1640-10% FBS medium. Forty thousand ACH2 cells per milliliter were induced with 5 ng of recombinant tumor necrosis factor alpha (TNF-α) (Sigma Chemical Co., St. Louis, Mo.) per ml for 24 hours. Twenty-four hours later, an equal volume of medium supplemented with 5 ng of TNF-α per ml and with the appropriate (2× final) concentration of the tested compound was added to cells. Viruses containing cell-free supernatants were collected 48 hours later, and they were subjected directly or after being pelleted through centrifugation for RT assay, p24 assay, and virus titration assay. Viability of the cultures was determined by XTT dye reduction). The RT assay, virus titration assay with MAGI cells, and p24 assay were performed as described above.

Pelleted virus particles were also subjected to Western blot analysis. The virion-associated viral proteins pelleted from 400 μl of cell free supernatant were resolved on 10% SDS-polyacrymide gels, were electroblotted onto polyvinylidene difluoride (PVDF) membranes, and were detected by AIDS patient sera or by rabbit-polyclonal anti-HIV-1 RT antibody (AIDS Research and Reference Program, Division of AIDS, National Institute of Allergy and Infectious Disease). Western blots were developed with standard methodology by chemiluminescence (Dupont-NEN, Wilmington, Del.) with a goat-anti human or goat anti-rabbit horseradish peroxidase-conjugated antibody (Bio-Rad, Hercules, Calif.).

### Example 8

This example describes molecular modeling that has been done concerning BAIPs. The following analysis was carried out on the HIV-1 RT coordinates 1RTH (Abola et al., 1987: Bernstein et al., 1977). A two-stage analysis was performed. First, the exterior surface of the HUV-1 RT heterodimer was probed for candidate binding regions. This process consists of localized sampling of the solvent accessible surface to determine a statistical probability that a candidate ligand may bind at this site. The model used to make the calculation has been parameterized, based on a broad sampling of protein-ligand crystal complexes available in the Brookhaven database of protein structures. (PDB) (Abola et al., 1987; Bernstein et al., 1977). The complete details for identification of putative protein binding sites can be found in Young et al. (Young et al., 1994). Second, the optimal docked position of the test ligand was determined. Families of possible conformations for the test ligand were generated using standard modeling techniques and each was docked to the regions defined in the first step. The docking procedure has been demonstrated to have an accuracy of within 1 Årms deviation from the known docked positions (Wallqvist & Covell, 1996). The position of the ligand with the strongest calculated binding strength is reported herein.

### Example 9

This example describes the preparation of samples for electron microscopy. Sample preparation for electron microscopy is described previously (Gonda et al., 1985). Briefly, the virus pellets were fixed with a 0. 1M sodium cacodylate buffer containing 1.25% glutaraldehyde, pH 7.2, followed by a 1% osmium tetroxide in the same buffer. The fixed pellets were dehydrated in a series of graded ethanol solutions (35%, 50%, 75%, 95% and 100%) and propylene oxide. The pellets were infiltrated overnight in an epoxy resin (LX-1 12) and propylene oxide mixture, then embedded in epoxy resin to cured for 48 hours at 60C. Thin-sections (50 to 60 nm) of the pellet were cut, mounted on a naked copper grid, and double stained with uranyl acetate and lead citrate. The thin sections were stabilized by carbon evaporation in a vacuum evaporator, observed, and photographed with a Hitachi H-7000 electron microscope operated at 75 kv.

The present invention has been described with respect to certain embodiments. The scope of the invention should not be limited to these described embodiments, but rather should be determined by reference to the claims.

### REFERENCES

1. Abola, E. E., et al. (1987). In Crystallographic Databases—Information Content, Software Systems, Scientific Applications, (Allen, F. H., Bergerhoff, G. & Sievers, R., eds), pp. 107-132.

2. Bernstein, F. C., et al. (1997) J. Mol. Biol. 112:535-542.

3. Baba, M., et al. (1989) Biochem. Biophys. Res. Comm. 165:1375-1381.

4. Balzarini, J. et al. (1992) Proc. Natl Acad. Sci USA. 89:4392-4396.

5. Buckheit, R. W. et al. (1997) Antimicrob. Agents Chemother. 41:831-837.

6. Bushman, F .D., and R. Craigie (1991) Proc. Natl. Acad. Sci. USA 88:1339-1343.

7. Ciminale,. V. et al. (1990) AIDS Res. Hum. Retrovir. 6:1281-1287.

8. Cohen, J. (1997) Science 277:32-33.

9. Cohen, K. A. et al. (1991) J. Biol. Chem. 266:14670-14674.

10. Condra, J. H. et al. (1992) Antimicrob. Agents Chemother. 36:1441-1446.

11. De Clercq, E. (1992) AIDS Res. Hum. Retrovir. 8:119-134.

12. De Clercq, E. (1996) Rev. Med. Virol. 6:97-117.

13. Debyser, Z. et al. (1991) Proc. Natl. Acad. Sci. USA. 88:1451-1455.

14. Finzi, D et al. (1997) Science 278:1295-1300.

15. Gao, H. Q., et al. (1998) J. Mol. Biol. 277:559-572.

16. Goldman, M. E. et al. (1991) Proc. Natl. Acad. Sci. USA. 88:6863-6867.

17. Goldman, M. E., et al. (1992) Antimicrob. Agents Chemother. 36:1019-1023.

18. Gonda, M. A., et al. (1985) Science 227:173-177.

19. Gu et al. (1993) J. Biol Chem 269:28119-28122.

20. Gao et al. (1998) J. Mol. Biol. 1998, 277: 559-572.

21. Howard, O. M. Z. et al. (1998) J. Med. Chem. 41:2184-2193.

22. Kimpton, J., and M. Emerman (1992) J. Virol. 66:2232-2239.

23. Koup, R. A. et al. (1991) J. Infect. Dis. 163:966-970.

24. Mellors, J. W. (1995) Intl. Antiviral News 3:8-13.

25. Merluzzi, V. J. et al. (1990) Science 250:1411-1413.

26. Miyasaka, T., H. et al. (1989) J. Med. Chem. 32:2507-2509.

27. Pauwels, R., K. et al. (1993) Proc. Natl. Acad. Sci. USA. 90:1711-1715.

28. Pauwels, R., K. et al. (1990) Nature 343:470-474.

29. Quinones-Mateu, M. E. et al. (1997) J. Virol. 29:364-373.

30. Rice, W. G. et al. (1997) Antimicrob. Agents Chemother. 41:419-426.

31. Rice, W. G., et al. (1993) Nature 361:473-475.

32. Rice, W. G. et al. (1995) Science 270:1194-1197.

33. Rice, W. G. et al. (1996) J. Med. Chem. 39:3606-3616.

34. Rice, W. G. et al. (1993) Proc. Natl. Acad. Sci. USA 90:9721-9724.

35. Rice, W. G. and J. P. Bader (1995) in Advances in Pharmacology (J. T. August, M. W. Anders, F. Murad and J. T. Coyle, eds) pp. 389-438.

36. Romero, D. L. et al. (1991) Proc. Natl. Acad. Sci. USA. 88:8806-8810.

37. Romero, D. L et al. (1993) J. Med. Chem. 36:1505-1508.

38. Ryabova et al.(1996) Pharm. Chem. J. 30:579-584.

39. Ryabova et al.(1996) Khim.-Farm. Zh.30:42-45

40. Ryabova et al. (1996) Mendeleev Commun. 3:107-109

41. Turpin, J. A. (1997) Antiviral Chem Chemother. 8:60-69.

42. Wallqvist, A. and D. G. Covell (1996) Proteins 25:403-419.

43. Weislow, O. S. (1989) J. Natl. Cancer Inst. 81:577-586.

44. Wishka, D. G. et al. (1998) J. Med. Chem. 41:1357-1360.

45. Wong, J. K. et al. (1997) Science 278:1291-1295.

46. Young, L. et al. (1994) Prot. Sci. 3:717-729.

47. Zack, J. A., et al. (1990) Cell 61:213-222.

We claim:

1. A compound having Formula I 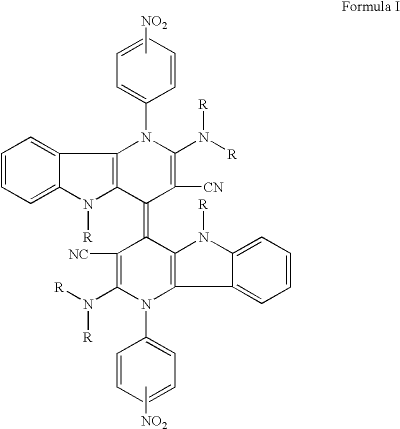
where R is selected from the group consisting of hydrogen and lower aliphatic.

2. The compound according to claim 1 where R is lower alkyl.

3. The compound according to claim 1 where R is methyl.

4. The compound according to claim 1 where the compound is

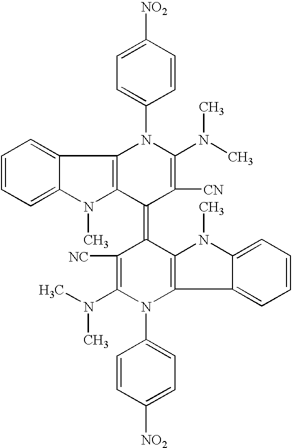

5. A method for treating a subject, comprising: providing a compound having Formula II 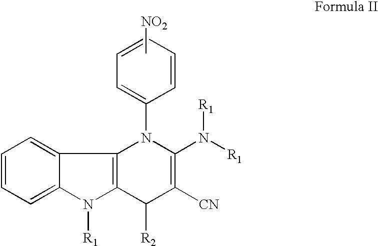
where R₁ is selected from the group consisting of hydrogen and lower aliphatic, and R₂ is selected from the group consisting of —CH₂COCH₃ and 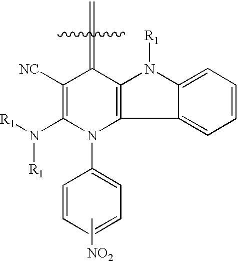
and
administering an effective amount of the compound to the subject.

6. The method according to claim 5 where R₂ is

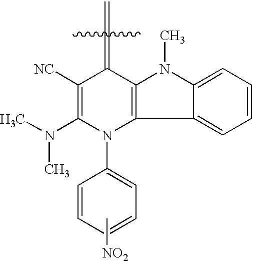

7. The method according to claim 5 where the compound is

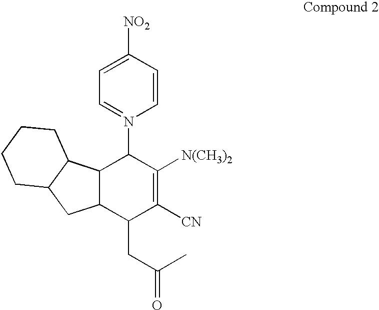

8. The method according to claim 5 where the compound is

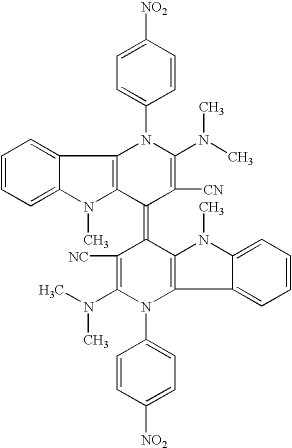

9. The method according to claim 5 where the subject is a mammal.

10. The method according to claim 5 where the subject is a human.

11. The method according to claim 5 where the effective amount is from about 0.1 mg/kg body weight per day, to about 200 mg/kg body weight per day, in single or divided doses.

12. The method according to claim 5 where administering comprises administering the compound topically, orally, intramuscularly, intranasally, subcutaneously, intraperitoneally, intravenously, or combinations thereof.

13. The method according to claim 5 where the compound is administered as a pharmaceutical composition.

14. A pharmaceutical composition comprising an effective amount of a compound having Formula 1 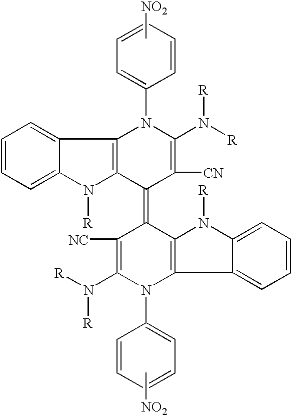
where R is selected from the group consisting of hydrogen and lower aliphatic.

15. The composition according to claim 14 where Ris lower alkyl.

16. The composition according to claim 14 where the compound is [chemical structure]
